(19) United States
(12) Patent Application Publication
(10) Pub. No.: US 2013/0078288 A1
(43) Pub. Date: Mar. 28, 2013

(54) Method of Treating Cancer

(71) Applicant: Chun Ho Yu, Hong Kong (HK)

(72) Inventor: Chun Ho Yu, Hong Kong (HK)

(21) Appl. No.: 13/681,438

(22) Filed: Nov. 20, 2012

Related U.S. Application Data
(63) Continuation-in-part of application No. 12/276,139, filed on Nov. 21, 2008, now abandoned.
(60) Provisional application No. 60/989,739, filed on Nov. 21, 2007.

(51) Int. Cl. A61K 47/12; A61K 31/337; A61K 33/24; A61K 31/045; A61K 33/26; A61K 9/14; A61K 45/06; A61K 31/704

(52) U.S. Cl. CPC A61K 47/12; A61K 45/06; A61K 31/337; A61K 31/704; A61K 31/045; A61K 33/26; A61K 9/14; A61K 33/24; B82Y 5/00
USPC 424/400; 514/784; 424/649; 514/449; 514/34; 514/724; 424/646; 977/811; 977/773; 977/915

(57) ABSTRACT

A method and composition for administering a therapeutic composition to a lesion comprising about 20% to about 50% ethanol and other novel therapeutic agents.

### CROSS-REFERENCE TO RELATED APPLICATIONS

This application is a Continuation-In-Part in accordance to MPEP 201.08 to Utility Application 12/276,139 filed on Nov. 21, 2008 which claimed the benefit of Provisional Application 60/989,739 filed on Nov. 21, 2007.

### FIELD OF THE INVENTION

This invention relates to the field of delivery and retention of cancer therapeutic agents in solid tumors.

### BACKGROUND OF THE INVENTION

### Description of the Prior Art

Throughout this disclosure, various publications and references by a number within parentheses. The full bibliographic citation for each reference can be found at the end of this application preceding the claims. The disclosures of these references are hereby incorporated by reference into this disclosure.

Hepatocellular carcinoma (HCC) is a common cancer that forms solid tumors in the liver. In some treatment methods, transarterial therapy such as transcatheter arterial chemoembolization (TACE) is a well-recognized procedure shown to have a significant impact on patient survival. HCC lesions are occasionally partially supplied at the periphery by the portal vein, especially when they are unencapsulated. The neoplastic tissue around the tumor boundary receives portal blood through the sinusoids communicating with its blood spaces (1). The portal contribution to tumor blood supply at least partially accounts for the fact that transarterial embolization or chemoembolization is often ineffective to control the tumor at the tumor periphery (2, 3).

Lipiodol, which is iodinized poppy seed oil, and typically used as a contrast medium, is capable of carrying drug formulations introduced transarterially. It is used in chemoembolization applications as an agent in follow-up imaging. Oftentimes, the formulation can be shunted away from the arterioles by peribiliary plexus (4, 5). The limitations that transarterial therapy are in need of improvement.

Lipiodol can be mixed with ethanol wherein the ethanol helps to solubilize drug formulations for transport to a solid tumor or lesion. A number of prior art discloses this use.

U.S. Patent Application 2006/0228304 discloses a liquid emulsion for radio imaging. The emulsion comprises a contrast medium, Lipiodol, and alcohol. The imaging agent is identified as Ethibloc, which is an occlusion emulsion containing zein, sodium amidotrizoate tetrahydrate, ethanol and other natural products.

U.S. Pat. No. 6,762,017 describes a simulated whole blood control mixture including an aliphatic alcohol and Lipiodol mixture. The mixture serves as a control for complete blood count analysis instrumentation.

U.S. Pat. No. 6,426,367 discloses methods and compositions for selectively occluding blood supplies to neoplastic tissue. The compositions include dilute mixture of ethanol and contrast/carrier agent in a saline solution. PUFA salt was dissolved in sterile sale, sterile phosphate buffered saline, or dilute ethanol in saline (final concentration <0.02% ethanol). The final concentration of PUFA in these solutions was approximately 25%. The PUFA solution was mixed with an iodized lymphographic oil (LIPIODOL FLUIDE®) in a ratio of 1:1.5 and 1:3 (volume/volume).

U.S. Pat. No. 6,878,688 describes methods for the treatment of malignant neoplasms using compositions comprising a chemotherapeutic agent dissolved in contrast/carrier agent and ethyl alcohol. Please note col. 1, 1. 63 to col. 2, 1. 2:
The closest method to the present method—the prototype—is a method of treatment of primary liver cancer, comprising injection of the preparation doxorubicin dissolved in contrast/carrier agent ultrafluid into the liver artery, while as the preparation doxorubicin doxorubicin-estrone dissolved in 96% ethyl alcohol at 70-76° C. in a dose of 20-60 mg in 10-15 ml of Lipiodol ultrafluid is used. 20 minutes prior to that a dose of 2-10 mg of AFP in 12-15 ml of physiological salt is injected into the liver artery; the repeated treatment is earned out after 3̂4 weeks (U.S. Pat. No. 2,065,307, cl. A 61 K 38/17, published Aug. 20, 1996, Bulletin No. 23).

The use of ethanol in the foregoing disclosures is for solubilizing the therapeutic agent and transporting it to the liver. There is no indication that ethanol, in specific doses, can be utilized as a therapeutic agent for the treatment of liver cancer. There is also no indication that ethanol, in specific doses, may be effectively combined with specific doses of contrast/carrier agents, fatty acid formulations, and/or other therapeutic agents to increase retention rate and ablate liver cancer.

There is a need to provide therapeutically active agents and inflammatory agents including ethanol to solid tumors for long retention periods, thereby improving the selectivity of the therapeutically active agent for the tumors and also reducing damage to normal tissues.

### SUMMARY OF THE PRESENT INVENTION

One aspect of the present invention is for the treatment of neoplastic areas or solid tumors by occluding the blood supply to the tumor by the administration of an iodinized poppy seed oil (Lipiodol) used as a contrast/carrier agent in this invention and moderate amounts of ethanol, about 20% to about 50% by volume in the poppy seed oil. Prior art references describe higher amounts of ethanol in poppy seed oil thereby causing unnecessary side effects. The carrier allows for favorable bio-distribution of therapeutic agents for cancer treatment and for the combination of therapeutic agents for enhanced treatment efficacy.

Another aspect of the present invention is to treat solid tumors by the administration of a fatty acid mixture, about 20% to about 50% by volume ethanol and/or other chemotherapeutic agents so that the agents and ethanol are retained in the solid tumors for long periods of time. The therapeutic agents in ethanol retained in the tumors occlude the vasculature of solid tumors. The specific amounts of ethanol and therapeutic agent also occludes the portal venous branches that supply the periphery of the solid tumors in the liver. Therefore another aspect of the present invention is for the treatment of hepatocellular carcinoma (HCC).

Another aspect of the present invention involves the use of paramagnetic, ferromagnetic and/or or biologically-inert metallic nanoparticles or microparticles. These particles can be activated by external fields of electromagnetic wave or magnetic field to a state of hyperthermia so that they serve as a form of localized hyperthermia therapy.

Another aspect of the present invention is to identify other ideal novel formulations/therapeutic compositions that include about 20% to about 50% ethanol by volume to increase retention rates and expedite the treatment of hepatocellular carcinoma (HCC).

It is another aspect of the present invention to provide radioisotopes, such as radioactive iodine 131 that can be mixed with contrast/carrier agent and localized in a solid tumor and be retained of the solid tumor for producing radioactivity for a selective internal radiotherapy.

### DETAILED DESCRIPTION OF THE PREFERRED EMBODIMENTS

As used herein, the term “contrast/carrier agent” means any of the class of compounds that are used, or may be used, to visualize lymphatics and lymph nodes, as well as veins and arteries, following an intravenous or intra-arterial injection. Lymphographic agents are typically vegetable oils (e.g., poppy seed oil), which are iodized (e.g., approximately 30 to 45by weight) and may be further derivatized (e.g., ethyl esterification). Examples include the iodized fatty acids of poppy seed oil LIPIODOL also commercially available as LIPIODOL ULTRA FLUIDE® from Laboratoire Guerbet, Paris., France, the ethiodized fatty acids of poppy seed oil (commercially available as ETHIODOL® from Savage Laboratories, Melville, N.Y.), and iophendylate (PANTOPAQUE® from Kodak). See, Horn et al. (1957), J. Am. Pharm. Assoc. Sci. Ed. 46:254; Paxton et al. (1975), Brit. Med. J. 1:120. As used herein, the term “contrast/carrier” means any agent that is useful for noninvasively visualizing blood vessels including, without limitation, radiography, CAT scans, MRI scans, ultrasound imaging, and the like. The preferred contract/carrier agent is Lipiodol. When recited in the present invention and mixed with ethanol, the compositions or mixture is identified as LEM. The combination carrier has the ability to accumulate within the vasculature of solid tumors. It has the ability to be retained within tumors for long periods of time and has a significantly higher degree of retention within tumors when compared to other formulations. These properties allow a localized and concentrated quantity of therapeutic agents to be retained within the tumor for a prolonged period of time and therefore enhance their therapeutic action. Types of agents that can be transported in the carrier include theromagnetic particles of the appropriate diameter for use in thermotherapy, chemotherapeutic drugs and radioactive therapeutic substances, for example, isotopes. The amount of ethanol in the contrast/carrier agent also serves as a therapeutic agent.

The prior art describes the use of ethanol as a carrier system for chemotherapeutic agents, such as doxorubicin. In the prior art references, ethanol is used in relatively small amounts for example up to 2%, as shown in U.S. Pat. No. 6,426,367. In accordance with the present invention, large amounts of ethanol are used with a contrast/carrier when the ethanol is itself a therapeutic agent. In a mixture with contrast/carrier agent, ethanol may be present in amounts ranging from about 20 to about 50% by volume to provide a therapeutic effect in treating neoplastic areas and solid tumors, particularly solid tumors in hepatocellular carcinoma (HCC). The therapeutic effect of LEM of the present invention is due to the embolization as well as ablative effect in HCC.

The retention time of a mixture of contrast/carrier agent and ethanol in solid tumors is extremely long. For example, the mixture may be retained in solid tumors for periods of up to about 15 months to about 51 months causing complete ablation or near complete ablation of a neoplasm or solid tumors. The contrast/carrier agent-ethanol mixture of the present invention has been found to be safe and effective for transarterial ethanol ablation of intrahepatic lesions of HCC. LEM may be retained in the tumor for long periods of time, e.g., up to six years.

In one typical procedure, 14 lesions were successfully treated with LEM. The treatment was followed with CT scanning for 15 months to 51 months, median 33.5 months, average 31.07±10.64 months, percentage of tumor volume reduction was found to be 21.82% to 93.18%, median 76.26%, average 72.07%±19.77%. Percentage of tumor tissue stained with the formulation was 36.67% to 93.33%, median 61.54%, average 62.24%±16.45%. Therefore, the formulation can be retained in 60% of the tumor tissue by volume for a median of 33.5 months.

Tumor size shrinks over time at a rate of 12% per month. In typical conditions, tumor shrinkage has been ⅛ of its original volume over a 16-month period.

In addition, the embolization efficiency and treatment efficacy of transarterial ethanol ablation (TEA) with the contrast/carrier agent-ethanol mixture of the present invention has proven to be superior to that of the transarterial catheter chemoembolization transarterial catheter chemoembolization (TACE).

The contrast/carrier agent-ethanol mixture of the present invention may also serve as carrier for other chemotherapeutic agents when an effective amount of the agent is used to treat a neoplasm or solid tumor. The chemotherapeutic agent may be one that is compatible with contrast/carrier agent and ethanol. The chemotherapeutic agents, separate from ethanol, may be doxorubicin, cisplatin, vincristine, adriamycin, taxol and other conventional chemotherapeutic agents. Therapeutic doses of chemotherapeutic agents, such as doxorubicin and cisplatin may range from 50 mg to 100 mg, depending upon the size of the lesion, can be mixed with LEM. LEM is delivered to the target organ/tissue/lesion through arterial blood vessels (transarterial delivery) and through catheters. LEM, while serving as a carrier for other therapeutic agents, will also increase the retention time of the therapeutic agent within the tumor lesion. It will also provide synergistic therapeutic action simultaneously.

Radioactive Lipiodol is produced by changing the iodine moiety to therapeutic radioactive iodine 131 (I-Lipiodol) using an atom to atom exchange reaction in a radioisotope laboratory. Lipiodol-iodine 131 is mixed with absolute ethanol in the ratio by volume of 2:1. It has a radiotherapeutic effect to malignant tumors when a therapeutic dose of iodine 131 up to 150 Gray is given to the tumor lesion, depending on the lesion size. The use of a mixture of Lipiodol-iodine 131 and ethanol greatly lengthens the local retention time of radioactivity with the lesion and enhances radiotherapeutic effect.

Magnetic nanoparticles including ferromagnetic and/or paramagnetic nanoparticles or microparticles may be mixed with LEM and delivered into tumor lesions transarterially. The particles are retained by LEM within the lesion and activated with alternative magnetic fields or other means to produce a thermal ablative effect.

The composition or mixture of the present invention is preferably administered intra-arterially to an artery which is proximal to the neoplastic region or solid tumor to be treated. Typically, the location of the region or tumor must be identified. This can be accomplished by a method as known in the art, for example, x-rays, computerized axial tomography (CAT) scans, or magnetic resonance imaging (MRI) scans. Contrast agents, such as contrast/carrier agent can specifically target the neoplastic tissues or solid tumors using selective catheterization of the respective arteries supplying the targets. The intra-arterial injection site is typically chosen to be close to or proximal to the solid tumor or neoplastic region to increase the portion of the dosage which reaches that region or tumor. The mixture of the present invention occludes the vasculature of the solid tumor, the arterial feeders, as well as the portal venous branches that supply the periphery of the solid tumor. The use of contrast/carrier agent aids in the visualization of the occlusion of the tumor feeding vessels.

Dosages of the mixture of the present invention depend primarily on the volume of the tumor and the degree of vascularization of the tumor. The dosage range may be an effective amount to occlude the vasculature. The amount of ethanol in the mixture ranges from 20% to about 50% by volume of the mixture. The total amount of the mixture of the present invention should not exceed 60 mL for each treatment. When additional chemotherapeutic agents are utilized with the contrast/carrier agent-ethanol mixture they may also show synergistic effect on the neoplastic area or solid tumor. The amount of the chemotherapeutic agent is an amount that is effective for cancer treatment or provide other action to destroy the tumor.

The contrast/carrier agent-ethanol mixture of the present invention when the ethanol present amount ranging from 20 to 50% by volume has shown superior reduction in tumor size in relatively short periods of time. For example, as tables show in Example 1, average reduction in tumor volume was 65.2% of the original volume.

### EXAMPLE 1

Transarterial Ethanol Ablation of Hepatomas with Lipiodol-Ethanol Mixture: A Prospective Phase-2 Study

Transarterial ethanol ablation of intrahepatic lesions of hepatocellular carcinoma (HCC) with transcatheter administration of contrast/carrier agent-ethanol mixture (LEM) enhances long-lasting embolization of both the arterioles and portal-venules supplying HCC, and therefore potentially a potent treatment.

Methods

Seventy-seven patients (60 men, 17 women, average age 63.4±11.3 years) were recruited and 164 intrahepatic lesions of HCC treated. The average size of the largest tumor lesion at enrollment was 5.2±3.0 cm, ranged 1.5 cm to 15 cm. LEM consisted of 33% ethanol by volume. Adverse events and laboratory test results were observed. Tumor response was evaluated with CT scan and serum alpha-fetoprotein (AFP) level. Patient survival was estimated with the method of Kaplan-Meier.

Results

The average number of treatments for each patient was 2.3±1.4 sessions. Acute hepatic decompensation and irreversible hepatic decompensation occurred in 9.9% and 0.6% of the procedures respectively. Complete ablation and near-complete ablation were noted in 86% and 12.8% of the 164 treated lesions respectively. Complete ablation by radiological criteria was achieved in 61 patients (79.2%). Tumor response as evaluated with serum AFP level showed a complete or near-complete response in 54% of patients. The median overall survival time was 2.2 years. Patient survival rate and progression free survival rate at 1 year, 2 years were 77.9%, 50.1% and 63.6%, 46.3% respectively.

Conclusion

Transarterial ethanol ablation is a safe and effective means for local control of intrahepatic lesions of HCC.

Materials and Methods

This was a prospective study with 77 patients. A total of 164 tumor lesions were detected and treated in this study, including 106 lesions detected at the time of patient enrollment and 58 lesions detected subsequently. The average number of lesions in each patient was 1.3±0.7, with a range of one to four lesions and a median of one lesion. The average size of the largest tumor lesion at enrollment was 5.2±3.0 cm, with a range of 1.5 cm to 15 cm and a median of 4.5 cm. The size of largest tumor lesion at enrollment is shown in Table 1.

TABLE 1
Size of Largest Tumor at Enrollment

| | | |
|---|---|---|
| Size | Frequency | Percent |

| | | | |
|---|---|---|---|
| ≦3 | cm | 20 | 25.97 |
| 3-5 | cm | 32 | 41.56 |
| ≧5-10 | cm | 17 | 22.08 |
| >10 | cm | 8 | 10.39 |

Formulation of Therapeutic Agent LEM formulation was prepared by drawing 2 ml of Lipiodol contrast/carrier agent (equivalent to ethiodol, ethyl ester of fatty acid of poppy seed oil; contains 37% by weight of iodine, Savage Laboratories, Melville, N.Y.); and 1 nil of absolute ethanol (99.9% ethyl alcohol; Quantum Chemical Corporation, Tuscola, Ill.) into 3 ml syringes that were vigorously shaken with a forward and backward motion for 20 cycles until a clear champagne-like homogeneous solution is formed. The mixture was prepared within five minutes of its administration into the patient.

Treatment Procedure.

The treatment procedure was performed under local anesthesia with 3 ml to 5 ml of 1% Lidocaine given at the groin and started with a hepatic arteriogram to identify the tumor. The most distal tumor-feeder or feeders accessible with a microcatheter, which could be subsegmental arteries or segmental arteries, were catheterized with a micro-guidewire and microcatheter system (Taper-16 Flex Tip Guidewire, FasTracker-18MX Infusion Catheter, Target, Boston Scientific, Fremont, Calif. 94538 USA). There was no limit to the number of tumor feeders selected. The number of feeders was usually less than six in a patient. Selective arteriograms were performed with the microcatheter to confirm full coverage of the tumor. The selected arteries were infused with 1% Lignocaine just before administration of LEM to prevent pain and vasospasm. The total dose of Lignocaine was within 5 ml in a patient. The vasculature of all part of the lesions was then completely filled up with LEM at a rate of 0.5 ml to 1 ml per minute until there was arterial backflow or appearance of portal venules. The volume of LEM to be delivered was not pre-decided, it was dependent on the amount of LEM uptake by the tumor vasculature. The upper limit of total volume of LEM to be delivered in one treatment session was set at 66 ml to limit the dose of ethanol to 22 ml. When necessary, 50 microgram of Fentanyl and 2 mg to 3 mg of Midazolam were given to the patient for pain relief. The patients remained bed-rest after the procedure and were discharged on the next day unless there were complications.

Criteria of the Endpoint of Treatment Procedure

The endpoint of the treatment procedure was to fill up the tumor vasculature completely with LEM, as well as the portal venules adjacent to the tumor margin and the tumor feeders leading to the tumors. The aim of each treatment session was to achieve the treatment endpoint within one session. When the volume of LEM required for treatment exceeds 60 ml, an additional procedure was performed within month to complete the treatment.

Tumor Response

Since there was no single effective and accurate means to assess tumor response to treatment, tumor response was evaluated with three different and complementary methods in this study. The first evaluation was based on radiological criteria as evaluated with CT scan. CT scan rather than magnetic resonance imaging was chosen for evaluation of tumor response because of convenience. CT scans were performed with the equipment LightSpeed 16 Plus of GE Medical System in the authors' institution, with a standardized technique using a setting of 5 on 5 mm, 120 KV, 300 mA, Pitch 0.938:1, 0.8 second/rotation. Contrast scan was performed with 100 ml intravenous Iomeron 250 given at a rate 2.5 ml/second, arterial phase at 30 second, porto-venous phase at 70 second, and delay phase at 300 second. The scans were interpreted centrally by two independent radiologists specialized in hepatic imaging. Pre-treatment CT with plain scan and triphasic contrast-enhanced scan of the abdomen was performed within one month before the LEM treatment to provide baseline information regarding the size and number of lesions. Plain CT of the liver was performed within one hour after intra-arterial administration of LEM to evaluate the adequacy of LEM coverage of the lesions by comparison with the pre-treatment scan. Follow-up CT with triphasic contrast-enhanced scan was performed at one month and two months after treatment, then two monthly up to 6 months after treatment, and then three monthly afterwards. The CT examinations were repeated with the same schedule for patients who underwent a repeat treatment. For patients who required two procedures to complete a treatment, the follow-up scans were started one month after the second treatment. The following were observed on follow-up CT: size of the treated tumors, evidence of new tumor lesions, residual or recurrent lesions of the treated tumors, evidence of intrahepatic venous invasion and extrahepatic metastasis.

The second evaluation was based on the percentage of greatest volume change of each individual treated lesion before and after treatment. The volume of each lesion was calculated with the formula ½ Width×½ Length×½ Depth×7i/3, where width, length, and depth were measured from CT.

The third evaluation was based on the response of scram AFP level to treatment. Serum level of alpha-fetoprotein (AFP) was examined before treatment and at each follow-up visit.

Results

Patient Selection

The diagnosis of HCC was confirmed with biopsy in 73 patients. In the other 4 patients, HCC was also confirmed in the specimen of previous liver resection and the new lesions under treatment showed typical CT and angiographic features of HCC. Nine of the 77 patients (11.7%) refused surgical resection. Surgical resection was contraindicated in the other 68 patients (88.3%). In this study, LEM treatment was the first treatment in 50 patients (65.9%), 27 patients (35.1% of 77 patients) had received a non-LEM treatment for HCC prior to selection and now presented with one or more new lesions typical of HCC, LEM was given to the new lesions as a salvage treatment for this group of patients.

Treatment Procedure The average number of treatment for each patient was 2.2±1.4 sessions, with a range of one to eight (one patient) sessions, and a median of two sessions. The average number of treatment given for each lesion was 1.4±0.9, with a range of one to five and a median of one. A total of 167 treatment procedures were performed. Thirty three patients received just one treatment. Only one treatment was given in 117 lesions, including 73 primary lesions and 44 secondary lesions. The total volume of LEM given in each patient ranged from 1 ml to 222 ml, with an average of 25.9±38.9 ml and a median of 12 ml. The average volume of LEM given in each treatment ranged from 0.45 ml to 63.5 ml, with a mean of 12.4±15 ml and a median of 6.5 ml.

TABLE 2
Number of LEM Treatment Sessions for
Individual Patients and Lesions

| Number of | Number of | Number of | Number of |
|---|---|---|---|
| Treatment | Patients | Original Lesions | Secondary Lesions |
| Sessions | (Total = 77) | (Total = 106) | (Total = 58) |
| 1 | 33 (42.8%) | 73 (68.8%) | 44 (75.9%) |
| 2 | 20 (25.9%) | 24 (22.6%) | 12 (20.7%) |
| 3 | 12 (15.6%) | 4 (3.8%) | 2 (3.4%) |
| 4 | 5 (6.5%) | 5 (4.7%) | 0 |
| 5 | 6 (7.8%) | 0 | 0 |
| 8 | 1 (1.3%) | 0 | 0 |

Tumor Response

Throughout the median follow up period of 2.3 years, an increase in number of tumor lesions was observed in 35 of 77 patients (45.45%) since the first treatment with LEM. Eleven patients had 1 new lesion, 11 patients had 2 to 3 new lesions, 6 patients had 4 to 5 new lesions, 2 patients had 6 to 9 new lesions, and 5 patients had 11 to 14 new lesions. The average number of lesions in each patient increased from 1.3±0_{—}7 at first treatment to 3.2±3.5. The maximum of number of lesions in each patient increased from 4 to 15. The median number of lesions in each patient increased from 1 to 2.

Tumor response as evaluated with radiological criteria is shown in Table 3.

TABLE 3
Treatment Sessions and Tumor-response by Radiological Criteria

| | | | No. of Lesions | No. of Lesions | No. of Lesions |
|---|---|---|---|---|---|
| | No. of | No. of | with Complete | with Near-complete | with Partial |
| | Treatment | Lesions | Ablation | Ablation | Ablation |
| | Sessions | Treated | (% of Total) | (% of Total) | (% of Total) |
| Original | 1 session | 77 lesions | 71 lesions | 6 lesions | 0 |
| Lesion | | | (92.2%) | (7.8%) | |
| | 2 to 4 | 29 lesions | 24 lesions | 5 lesions | 0 |
| | sessions | | (82.7%) | (17.3%) | |
| Secondary | 1 session | 41 lesions | 39 lesions | 0 | 2 lesions |
| Lesions | | | (95.1%) | | (4.9%) |
| | 2 to 3 | 17 lesions | 7 lesions | 10 lesions | 0 |
| | sessions | | (41.2%) | (58.8%) | |
| Total | 1-4 sessions | 164 lesions | 141 lesions | 21 lesions | 2 lesions |
| Lesions | | | (86.0%) | (12.8%) | (L2%) |

A reduction in tumor volume was observed in 112 of 164 lesions (68.3%). In 47 lesions (28.6%), there was no change in tumor volume after treatment. Increase in tumor volume was noted in 5 lesions (3%). Average tumor volume reduction was 65.2%, with volume reduction ranged from 2% to 97% and a median of volume reduction being 67.6%. There were 26 cases of vascular or biliary invasion or extrahepatic spread, including one case of malignant biliary obstruction, 11 cases of portal vein invasion, 2 cases of hepatic vein invasion, 5 cases of lung metastasis, 4 cases of adrenal metastasis, and 2 cases of lymph node metastasis. Bone metastasis did not occur.

Patient Outcome

Complete ablation by radiological criteria was achieved in 61 patients (79.2%). Three patients of this group went on to curative liver resection after successful down-staging of the liver tumor. One patient of this group received liver transplantation. Histological examination of the liver specimen in these 4 patients showed 85% to 100% necrosis of the treated tumors. Twelve patients subsequently developed progressive disease. In the other 16 patients with near complete or partial ablation (20.7%), 8 patients were treated with either local ablation or conservatively because the residual lesions were small and static, the other 8 patients developed progressive disease. In the 20 patients with progressive disease, progressive multifocal disease or venous invasion was seen in 16 patients, evidence of extrahepatic disease was seen in 9 patients. Two patients with progressive disease received systemic chemotherapy. The other patients were treated conservatively for various reasons such as impaired liver function, impaired renal function, deteriorated general condition, or patient's wish to give up further treatment.

The median follow up time of the whole cohort was estimated to be 2.3 years, with a range of 2.1 and 2.5 years at 95% confidence interval. The median overall survival time was 2.2 years, with a range of 1.5 and 3.1 years at 95% confidence interval. The estimated patient survival rate at 1 year and 2 year was 77.9% and 50.1% respectively. The estimated progression free survival rate at 1 year and 2 years were 63.6% and 46.3% respectively. [59] Transcatheter arterial embolization or intra-arterial injection of absolute ethanol as a treatment for HCC has been reported in previous studies (5, 6). Park et al performed super selective transcatheter arterial embolization with LEM of 75% by volume of absolute ethanol, to treat 14 patients with HCC lesions of size less than 5 cm and with prominent feeding artery, and found it a safe treatment for small nodular HCC, able to cause total or subtotal necrosis of tumor and thickening of capsule (5). Ito et al found that intra-arterial injection of 50% by volume of absolute ethanol was effective to achieve emergency hemostasis of HCC in 5 patients with ruptured HCC, and to prevent tumor rapture in 42 patients with impending rupture of HCC.

Transarterial ethanol ablation (TEA) of HCC with LEM as demonstrated in the present study represents a treatment concept different from that of transcatheter arterial embolization of HCC with absolute ethanol as reported in previous studies (5, 6). Instead of using LEM with a high proportion of absolute ethanol to contrast/carrier agent by volume as it was in those previous three studies (5, 6, 7), the formulation of LEM used in the present study consisted of a lowered proportion of ethanol to 33% by volume. Based on the studies of Kan et al. (4), LEM with a reduced ethanol composition has been shown to associate with a diminished degree of endothelial damage of the arterial feeder of tumor, and thereby facilitates effective delivery of LEM to tumor vasculature. The present study was also different from the other previous studies in that not just patients with small HCC lesions were enrolled, patients had been enrolled consecutively such that patients with large lesions of size up to 12 cm were included. Evaluation of treatment efficacy of TEA with LEM according to size ranges of tumor would be useful to guide patient selection, such evaluation would be covered by the authors in another article on factors affecting treatment outcome, otherwise it will add further length to the present over-length manuscript.

There are two potential advantages of TEA with LEM over TACE as a treatment for HCC. First, TEA with LEM is theoretically more effective to eradicate tumor cells at the periphery of a tumor that is supplied by portal venules, since TEA with LEM has been proved to be able to induce a long-standing embolization of both arterials and portal venules in the liver (4, 8). Although HCCs are supplied essentially by the hepatic arteries, it is known that well differentiated small HCCs are occasionally partially supplied by the portal vein, especially when there is a lack of capsule formation around the tumor (9, 10). To achieve complete necrosis of HCCs with portal supply, simultaneous and complete blockage of feeding arterioles and peripheral portal venules surrounding the tumors, including the peribiliary plexus was necessary and could be provided by TEA with LEM. Increased effectiveness of tumor eradication theoretically would reduce the need for repeat treatment and therefore reduce the risk of hepatic decompensation. Second, embolization of the feeding hepatic artery with gelatin sponge in TACE may lead to blockage of the feeding artery and development of complicated collateral supply to the tumor, making it difficult or impossible to perform a repeat TACE effectively (11, 12). Without the need for gelatin sponge embolization in TEA with LEM, the feeding hepatic artery is always preserved as shown in the current study, to allow for repeat treatment for residual or recurrent disease. The low incidence of post-embolization syndrome and absence of significant adverse effects on the gastrointestinal tract or biliary tract associated with LEM treatment as observed in the current study was in keeping with the findings of previous studies in which higher proportions of ethanol was used.

Assessment of change in tumor dimension based on the WHO criteria may not truly reflect tumor response to transcatheter embolization or local ablation in solid tumors such as HCC, because after such treatments a mass of dead tissue always remains even after complete eradication of tumor cells in the tumor mass, and it may take years for the dead-tissue mass to be completely resolved. Evidence to such postulation could be found in the current study. Despite the fact that the tumors in 18 patients were probably completely destroyed with LEM treatment, as indicated by normalization of the serum AFP levels, the “tumor masses” of these 18 patients remained present for years, although they gradually shrank down significantly in size. Serum AFP was a sensitive indicator of residual disease for those with an elevated basal level. Lesion enhancement suggestive of residual lesion on CT was always associated with recurrent elevation of AFP level. Recurrent rise in AFP may occur in patients with constant tumor size, indicating that unchanged tumor size did not preclude the presence of residual tumor. The high effectiveness and potency of TEA with LEM in local eradication of intrahepatic lesions of HCC was shown in the results of local tumor response to treatment as evaluated with radiological criteria and volume change of tumor lesions. Complete ablation and near-complete ablation were achieved in 86% and 12.8%, respectively of 164 lesions. Further evidence of the effectiveness of LEM treatment was reflected in the high complete-ablation rate of tumors in 79.9% of 77 patients and high AFP response rates of 68% in 50 patients, including major or better responses. The estimated 1 year and 2 year survival rate were 77.9% and 50.1%, superior to the survival rates of patients in a similar population with similar tumor size treated with TACE, 1 year survival 53% and 57% (4, 14), 2 year survival 31% and 38% (4, 14).

Transcatheter arterial embolization with Lipiodol (iodized oil)-ethanol mixture (LEM) has also been shown to be an effective treatment for intrahepatic lesions of HCC although it has been much less commonly known or used. Ethanol produces long-lasting embolization effect by causing endothelial damage and thrombosis of the arteriolar lumen of tumor feeders and tumor vasculature, and thereby leads to infarction of the tumor. Moreover, transarterial ethanol also causes embolization of the portal venules by way of the peribiliary plexus. LEM is potentially a more potent embolization agent for hypervascular tumors than other ethanol-free Lipiodol formulations. LEM treatment is also known to be a clinically safe procedure that is associated with a lowered incidence of post-embolization syndrome as compared to TACE, and absence of significant adverse effects in the gastrointestinal tract or biliary tract. In the next example, the bio-distribution properties of three transarterial Lipiodol-based therapeutic regimens, including LEM, pure Lipiodol, and Lipiodol followed by gelfoam embolization, were compared and evaluated in an in-vivo environment of human HCC. It was hypothesized that LEM is associated with a longer-lasting embolization effect for intrahepatic lesions of HCC, without significant increase in degree of lung shunting or decomposition rate, when compared with other ethanol-free Lipiodol formulations.

### EXAMPLE 2

### A Comparison of 3 Transarterial Lipiodol-Based Formulations for Hepatocellular Carcinoma: In-Vivo Bio-Distribution Study in Human

This study aims to compare the bio-distribution properties of three transarterial contrast/carrier agent-based therapeutic regimens in human hepatocellular carcinoma (HCC).

In this prospective study with 13 patients randomly allocated to one of three study groups, each of the patients received transcatheter intra-arterial administration into a solitary HCC with one of three different contrast/carrier agent-based formulations: contrast/carrier agent-ethanol mixture (LEM) of the present invention (Group A); Lipiodol contrast/carrier agent alone (Group B); and Lipiodol contrast/carrier agent and gelatin pledgets (Group C). With the use of radioactive iodine-131-labeled Lipiodol contrast/carrier agent, each group was assessed for: (1) pattern of contrast/carrier agent accumulation in the lungs within the first two weeks as evaluated with SPECT; (B) decomposition of contrast/carrier agent formulation within the first two weeks as evaluated with radioactivity detected in peripheral blood and urine; and, (3) degree of contrast/carrier agent retention in the tumor within the first four weeks as evaluated with CT.

Results

No statistically significant difference was detected in contrast/carrier agent accumulation in the lungs among all three groups. However, the peak accumulation in the lungs was delayed 3 days for Group A. The degree of contrast/carrier agent retention within the tumor in Group A was significantly greater than that in Group B and Group C on day 14 (p=0.014) and day 28 (p=0.013).

Conclusion

LEM is associated with a greater embolic effect, and comparable degree of lung shunting and decomposition rates, when compared with ethanol-free contrast/carrier agent formulations.

Materials and methods

Thirteen consecutive patients who fulfilled all of the following selection criteria were recruited: (1) unresectable tumor due to unfavorable liver size or tumor location; (2) histologically proven HCC; (3) solitary intra-hepatic tumor of massive expansive morphology and diameter between 4 to 6 centimeters as depicted on triphasic contrast-enhanced CT scan, only subjects with solitary lesions were selected in order to facilitate standardization of the procedure of transarterial infusion and simplification of data analysis; (4) hypervascular tumor as depicted on CT scan, defined as contrast enhancement in greater than 90% of the tumor volume as depicted in the arterial phase; (5) no CT evidence of tumor invasion of the hepatic vein or the portal vein; (6) no angiographic evidence of arterio-portal shunt or arterio-venous shunt associated with the tumor; (7) cirrhosis status of grade A by Child-Pugh classification, cirrhosis confirmed with needle biopsy; (8) patient being Hepatitis B virus carrier; (9) patient has not received any other treatment for HCC; (10) patient with normal renal function. The study procedure was commenced at 5 weeks before the patients were treated with transcatheter arterial embolization.

The 13 patients were randomly allocated to one of three study groups, in which the patients received transcatheter intra-arterial administration into the liver tumor with one of three different Lipiodol contrast/carrier agent-based formulations: Group A, Lipiodol contrast/carrier agent-ethanol mixture (LEM), Group B, Lipiodol contrast/carrier agent alone, Group C, Lipiodol contrast/carrier agent followed by gelatin pledgets. Since LEM was considered as a therapeutic formulation, no further treatment was given for Group A patients after LEM administration. For Group B and Group C patients, a follow up treatment with transcatheter intra-arterial LEM administration was given one month after the initial administration of contrast/carrier agent formulation.

Randomization was by drawing consecutively labeled and sealed envelopes that contained information on computer-generated group allocation. Five patients were allocated to Group A, four patients to Group B and four patients to Group C. The size of tumor ranged from 4 to 6 cm, with an average of 5.06 0.59 cm. The size of tumors in Group A ranged from 4.5 cm to 6.0 cm, with an average of 5.08±0.57 cm. The size of tumors in Group B ranged from 4.4 to 5.7 cm, with an average of 5.02±0.62 cm. The size of tumors in Group Cranged from 4.3 cm to 5.8 cm, with an average of 5.10±0.76 cm.

Pattern of Lipiodol contrast/carrier agent accumulation in the lungs within the first two weeks, decomposition of contrast/carrier agent formulation within the first two weeks, and degree of contrast/carrier agent retention within the tumor in the first four weeks were assessed in all three groups. The patients were discharged from the hospital after the first week.

Degree of Lipiodol Contrast/Carrier Agent Retention in the Tumor

The tumor in each of the patients was filled up with intra-arterial administration of one of the three contrast/carrier agent-formulations, with selective catheterization of the segmental or sub-segmental branch under fluoroscopic control, until the tumor vasculature could not admit any more, as indicated by stasis of the agent on fluoroscopy. A plain CT scan was performed for the patients of all three groups on day 0, day 14, and day 28, with slice thickness of 3mm, slice interval 3mm, and pitch 2.1. The quantity of contrast/carrier agent retention within the tumor at the three time points was measured in cubic millimeters from the volumetric CT data with a CT workstation (Advantage window 4.2, Lightspeed 16 plus. General Electric Medical System). For each tumor, the initial quantity of contrast/carrier agent accumulation within the tumor measured on day 0 was taken as the baseline value, the quantity of contrast/carrier agent accumulation measured on day 14 and day 28 respectively was taken as the retention value. The degree of contrast/carrier agent retention in each tumor was defined as the ratio of the retention value to the baseline value. The degree of contrast/carrier agent retention in three groups was compared. The degree of contrast enhancement in the treated tumors was not evaluated in the present study since evaluation of residual or recurrent tumor after treatment was not an objective of the present study.

Results

Procedure

Digital subtraction angiography (DSA) in the 13 patients showed that there was no evidence of arterio-portal shunt or arterio-venous shunt associated with the tumors. The arterial catheterization and embolization procedures were performed uneventfully without complications in all patients.

Pattern of Lipiodol Contrast/Carrier Agent Accumulation in the Lungs as Evaluated with SPECT

The quantity of contrast/carrier agent accumulation in the lungs is represented by the detected activity in the lungs on SPECT. The quantity of contrast/carrier agent accumulation in the lungs of each patient at the specified time points is represented as a percentage of the quantity at time of peak accumulation in the lungs.

In all three Groups, there was an initial high quantity of contrast/carrier agent accumulation in the lungs, followed by a gradual decline in amount of contrast/carrier agent afterwards. However, there was a difference between Group A and the other two groups regarding the pattern of change in contrast/carrier agent accumulation in the lungs over the various time points. In Group A, the quantity of contrast/carrier agent accumulation rose from day 0 for three days to reach a peak on day 3, whereas the peak occurred on day 0 for Group B and Group C. The maximum degree of contrast/carrier agent shunting to the lung m each patient was on average 18.49±4.24%, 18.89±10.05, and 18.85±6.89 in Groups A, B, C respectively. There was no significant difference in the degree of maximum lung shunting among the three groups.

Lipiodol Contrast/Carrier Agent Decomposition as Detected in Blood and Urine

The proportion of contrast/carrier agent that was decomposed over time within the first two weeks, is represented by the ratio of radioactivity detected in the peripheral blood to the total initial radioactivity administered. The proportion of contrast/carrier agent decomposed and passed out over time within the first week, is represented by the ratio of radioactivity passed out and detected in 24 hours urine specimen to the total initial radioactivity administered. For the analysis of the radioactivity of both blood and urine, multilevel modeling revealed that there was a linear trend across time (fixed effect p<0.001). In addition, the rate of change on kinetic was linearly varied among individuals (random effect p<0.001), indicating that there was no significant difference in the rate of contrast/carrier agent decomposition among the three groups.

Degree of Lipiodol Contrast/Carrier Agent Retention in the Tumor

The degree of Lipiodol contrast/carrier agent retention within the tumor in the first four weeks for the three different Lipiodol contrast/carrier agent formulations was determined. The degree of contrast/carrier agent retention within the tumor in Group A is shown to be significantly greater than that in Group B and Group C (p=0.014) on day 14. Post hoc comparison on day 14 indicated a greater degree of retention in Group A versus Group B (p=0.016), a greater degree of retention in Group A versus Group C (p=0.016), and no difference in degree of retention between Group B and Group C (p=0.686), with the level of significance of 0.05 adjusted by Bonferroni correction to 0.017 (0.05/3). The degree of contrast/carrier agent retention within the tumor in Group A is also shown to be significantly greater than that in Group B and Group C (p=0.013) on day 28. Post hoc comparison on day 28 indicated a greater degree of retention in Group A versus Group B (p=0.016), a greater degree of retention in Group A versus Group C (p=0.016) and no difference in degree of retention between Group B and Group C (p=0.886), with the level of significance of 0.05 adjusted by Bonferroni correction to 0.017(0.05/3).

Based on the bio-distribution evidence of the current study, LEM has favorable embolization properties when compared to those of the other ethanol-free formulations, while it is not associated with untoward bio-distribution properties when compared to the other ethanol-free formulations. Knowing that intra-arterial LEM treatment was shown to be a clinically safe procedure which is associated with a lowered incidence of post-embolization syndrome as compared to TACE (13), and absence of significant adverse effects in the gastrointestinal tract or biliary tract (13, 14). Treatment may be associated with potential clinical advantages such as less systemic chemotoxicity, less severity of post-embolization syndrome, and less costly. Combining chemotherapeutic agents with LEM increases the efficacy of LEM treatment.

This in-vivo bio-distribution study of three transarterial contrast/carrier agent-based therapeutic regimes showed that LEM has a greater embolic effect in intrahepatic HCC at 4 weeks, as indicated by a higher degree of Lipiodol contrast/carrier agent retention, without increase in the degree of lung shunting or decomposition rate, when compared with the other ethanol-free contrast/carrier agent formulations.

### EXAMPLE 3

Long Term Embolization Effect of Transarterial Ethanol Ablation with Lipiodol-Ethanol Mixture on Hepatocellular Carcinoma

The study aims to evaluate the embolization effect of transarterial ethanol ablation (TEA) with contrast/carrier agent ethanol mixture (LEM) on intrahepatic lesions of hepatocellular carcinoma (HCC) in terms of tumor regression and contrast/carrier agent retention.

Materials and Methods

Eleven intrahepatic HCC lesions in 9 patients were successfully treated with TEA with LEM and followed up regularly with triphasic CT scans at three months intervals. The criteria for successful treatment were 1) only one treatment was given, 2) no CT evidence of contrast enhancing focus within the lesion at arterial phase, 3) CT evidence of continual shrinkage of the lesion. Tumor regression rate was evaluated with the percentage reduction in tumor volume over time. Embolization effect was evaluated with the percentage retention of contrast/carrier agent within tumor over time.

Results

The patients were followed up for an average of 795.2±445.4 days, median 590 days, range 251 days to 1541 days. There was continual reduction in volume of all 11 tumors. At the last follow scan, the average volume of tumor was 21.3±16.8% of the original tumor volume, median 17.2%, range 1.6% to 60.2%. The average proportion of contrast/carrier agent-stained tissue within the tumor lesion was 91.1±6.3%, median 91.5%, range 79.9% to 100%.

Conclusion

Successful embolization of intrahepatic lesion of HCC with LEM is always associated with a continual and high degree of tumor volume regression, and also a high degree of contrast/carrier agent retention with the tumor.

### EXAMPLE 4

### Superior Embolization Efficacy and Treatment Efficacy of Transarterial Ethanol Ablation for Hepatocellular Carcinoma as Compared to Transcatheter Arterial Chemoembolization

The aims of this study are to evaluate in general the correlation between embolization efficacy and treatment efficacy in transarterial treatment of hepatocellular carcinoma (HCC), and to compare the embolization efficacy and treatment efficacy of transarterial ethanol ablation (TEA) with contrast/carrier agent-ethanol mixture (LEM) to that of transarterial catheter chemoembolization (TACE).

Method

This is a prospective case-control study with 30 patients in each of the two groups (LEM, TACE) matched with the following criteria: Child A cirrhosis, solitary tumor, size within 12cm diameter. LEM contains 33% by volume of absolute ethanol. TACE was performed with contrast/carrier agent-cisplatin emulsion and embolization with gelatin sponge pledgets. Embolization efficacy was assessed with the degree of contrast/carrier agent retention (DLR) within the tumor at 2 month after a single initial treatment. Treatment efficacy was assessed with overall survival (OS), intrahepatic (IDP) and extrahepatic disease progression rate (EDP), and progression free survival rate (PFS).

Result

For the whole group, when patients with DLR<60% at two months was compared to patients with DLR>60%, one year OS was 66.7%, 88.9% (P=0.0192), one year IDP was 59.4%, 25.6% (P=0.0169), one year EDP was 35.5%, 0.31% (P=0.0047), one year DFP was 36.3%, 72.1% (P=0.005). DLR at two months was 89.5±10.7% (LEM group) and 47.5±1.2% (TACE group) P<0.0001). Overall survival rate at one year and two year were 93.3%, 80.0% (LEM group) and 73.3%, 43.3% (TACE group) (P=0.0053). Progression free survival rate at one year and two year were 69.8%, 58.8% (LEM group) and 46.0%, 42.5% (TACE group).

Conclusion

Embolization efficacy is a useful indicator of treatment efficacy. The embolization efficacy and treatment efficacy of TEA with LEM is superior to that of TACE.

### EXAMPLE 5

Embolization and Treatment Efficacy of Transarterial Therapy for Unresectable Hepatocellular Carcinoma: a Case-Controlled Comparison of Transarterial Ethanol Ablation with Lipiodol-Ethanol Mixture versus Transcatheter Arterial Chemoembolization

The aims of this study were to compare the embolization efficacy and treatment efficacy of transarterial ethanol ablation with Lipiodol-ethanol mixture to that of transcatheter arterial chemoembolization, and to evaluate the correlation between embolization efficacy and treatment efficacy in transarterial treatment of hepatocellular carcinoma.

Method:

This was a prospective case-controlled study with 30 patients in each of the two groups (Lipiodol-ethanol, chemoembolization) matched with the following criteria: Child-Pugh Classification, solitary tumor, size within 12 cm diameter, Eastern Cooperative Oncology Group Performance Status grade 0 or 1. The primary endpoints were embolization efficacy (defined as the degree of Lipiodol retention within the tumor at 2 month) and treatment efficacy as evaluated by tumor response (RECIST criteria), 1 year and 2 year intrahepatic and extrahepatic disease progression rates, progression free survival rate, and overall survival rate. The secondary endpoint was the correlation between embolization efficacy and treatment efficacy in terms of patient outcome.

Result:

When comparing Lipiodol-ethanol to chemoembolization, the degree of Lipiodol retention was significantly higher in Lipiodol-ethanol group than chemoembolization group (89.535 10.7% versus 47.5±21.2%, P<0.0001). Chance of lesion-size progression by RECIST criteria at 1 year was significantly higher in chemoembolization group (5 in 30, versus 0 in 30) (P=0.0261) The 1 year and 2 year overall survival in Lipiodol-ethanol group (93.3%, 80.0%) were significantly higher than those in chemoembolization group (73.3%, 43.3%) (P=0.0053). The 1 year and 2 year extrahepatic disease progression of Lipiodol-ethanol group (0%, 0%) were significantly lower than those in chemoembolization group (35.5%, 39.2%) (P=0.0002). There was no statistically significant difference in progression-free survival rate and in intrahepatic disease progression at 1 year and 2 year between the two groups

Patients with a higher Lipiodol retention (>60%) were associated with significantly better treatment outcome at 1 year than those with Lipiodol retention □ 60%, in terms of a higher overall survival (88.9% versus 66.7%, P=0.0192), a lower intrahepatic disease progression (25.6% versus 59.4%, P=0.0169), a lower extrahepatic disease progression (0.31% versus 35.5%, P=0.0047), and a higher progression free survival (72.1% versus 36.3%, P=0.005).

Conclusion:

Our findings suggest that the embolization efficacy and treatment efficacy of transarterial ethanol ablation with Lipiodol-ethanol mixture of the present invention is superior to that of chemoembolization for the treatment of patients with unresectable HCC. Embolization Efficacy is a useful early predictor of treatment efficacy.

Introduction

Hepatocellular carcinoma (HCC) is one of the most common solid malignancies in the world, with a rising incidence the United States and other developed western countries (1, 2). In many centers, transcatheter arterial chemoembolization, a common and well-recognized procedure shown to have a significant impact on patient survival (3, 4), is a mainstay of treatment for patients in whom other curative treatment-options are not applicable. Other options of transarterial treatment for HCC include bland embolization (5) and radioembolization (6). Transcatheter arterial embolization with Lipiodol-ethanol mixture has been shown to be an effective treatment for intrahepatic lesions of HCC although it is not widely known or used (7-9).

Transarterial ethanol ablation of HCC utilizing Lipiodol-ethanol mixture consisting of a lowered proportion of ethanol (33% by volume) represents a treatment concept different from that of transcatheter arterial embolization of HCC with a high proportion of absolute ethanol. Lipiodol-ethanol with a reduced ethanol composition has been shown to associate with a diminished degree of endothelial damage of the arterial feeder of tumor, and thereby facilitates effective delivery of Lipiodol-ethanol to arterioles such as tumor vasculature (10, 11). Ethanol produces long-lasting embolization effect by causing endothelial damage and thrombosis of the arteriolar lumen of tumor vasculature, thereby leads to infarction of the tumor. Such Lipiodol-ethanol formulation has been found to be safe and effective for treating HCC, especially for small lesions in patients with Child-Pugh Class A (12, 13). Because embolization is a vital component in both chemoembolization and the Lipiodol-ethanol treatment, an understanding of embolization efficacy in these treatments will allow one to evaluate the efficacy of these treatments. The aims of this study are to compare the embolization efficacy and treatment efficacy of transarterial ethanol ablation with Lipiodol-ethanol mixture to that of transcatheter arterial chemoembolization for the treatment of patients with unresectable HCC, and to evaluate the correlation between embolization efficacy and treatment efficacy in transarterial treatment of patients with unresectable HCC.

Materials and methods

This was a prospective case-controlled study of 60 patients with unresectable HCC treated with transarterial therapy. A consecutive group of patients receiving Lipiodol-ethanol treatment between March to December in 2002 was compared with another group receiving chemoembolization in 2005 were compared with each other. Starting from 2005, patients with HCC were treatment with TACE for which IRB approval was not obtained because TACE has been considered as a standard treatment at that time. The Lipiodol-ethanol group consisted of 30 consecutive patients with 1) histologically proven HCC, 2) Child A or B liver cirrhosis, 3) solitary tumor of diameter within 12 cm, 4) Eastern Cooperative Oncology Group Performance Status (ECOG) grade 0 or 1. The chemoembolization group consisted of 30 consecutive patients with liver and tumor characteristics matched with those of the Lipiodol-ethanol group. There was no statistical difference between the two groups concerning patient age, sex ratio, tumor size, Child-Pugh Classification of severity of liver disease, and ECOG performance status (table 1).

The primary endpoints of this study were embolization efficacy and treatment efficacy. Embolization efficacy was defined as the effectiveness in obliterating tumor vasculature as represented by the degree of Lipiodol retention within the tumor at 2 month after a single initial treatment. Treatment efficacy was defined as the effectiveness in achieving desirable treatment outcome as evaluated by tumor response and patient outcome. The secondary endpoint was correlation between embolization efficacy and treatment efficacy in terms of patient outcome in the whole group of 60 patients.

Parameter of Embolization Efficacy

The degree of Lipiodol retention was used as a parameter of embolization efficacy. Degree of Lipiodol retention was defined as the ratio of the volume of tumor tissue that was stained with Lipiodol at two months after the first treatment to the volume of Lipiodol-stained tumor tissue on the day of the first treatment. Non-contrast CT scan of the liver was performed on the day of the first treatment and at two months after treatment for detection of Lipiodol-stained tumor. The volume of Lipiodol-stained tumor was acquired from the CT Workstation by selecting an intensity threshold of 100 Hounsfield Unit. Collection of CT data on the volume of Lipiodol-stained tumor tissue was handled by an independent radiologist.

Parameters of Treatment Efficacy

Treatment efficacy was evaluated with tumor response and patient outcomes. Tumor response in each patient was assessed with triphasic CT scan for the tumor lesions that had been treated since the first treatment session. Tumor response was evaluated with the Response Evaluation Criteria in Solid Tumors (14) on the 12-months CT or the last CT before the death of the patient, whichever the earlier. Response of tumor lesions to treatment was classification into four categories: 1) complete response (absence of lesion), 2) partial response (lesion-diameter reduction by >30%), 3) static disease, 4) lesion-size progression (lesion-diameter increase by >20%). Patient outcome was evaluated with intrahepatic and extrahepatic disease progression rate at 1 and 2 years, progression free survival rate at 1 and 2 years, and overall survival rat at 1 and 2 years. Intrahepatic disease progression was defined as the occurrence of any new lesion after the onset of treatment as depicted on a triphasic CT scan. Extrahepatic disease progression was defined as the occurrence of CT evidence of extrahepatic disease detected after the onset of treatment, including intrahepatic venous invasion or biliary invasion.

Treatment Procedure

The Lipiodol-ethanol formulation was prepared by mixing two portions by volume of Lipiodol to one portion by volume of ethanol to form a clear champagne-like homogeneous solution. Transarterial delivery of Lipiodol-ethanol into tumor vasculature was performed via catheterization of the tumor feeder or feeders with a 5 French catheter or a micro-catheter. Lipiodol-ethanol was infused until there was flow stagnation. An average of 14.5±17.6 ml of Lipiodol-ethanol was given for the first treatment of each patient in the Lipiodol-ethanol group. Each patient received a median of 2 treatments and a range of one to five treatments throughout the follow up period. Typically the amount of Lipiodol-ethanol given in repeat treatments was much less than that of the initial treatment. Technical details of the Lipiodol-ethanol treatment were described by Yu (15).

A standard dose of 20 ml of Lipiodol-cisplatin emulsion containing 10 mg of cisplatin was infused into the feeder or feeders of the tumor that was catheterized with a 5 French catheter or a microcatheter. Gelfoam embolization was performed afterwards until there was flow stagnation at the feeding hepatic artery. Each patient received a median of 3 treatments and a range of one to six treatments throughout the follow up period.

The treatment goal in both groups was delivery of the Lipiodol-based therapeutic agent to fill up tumor vasculature. The goal was achieved typically by lobar or segmental catheterization depending on the size of the lesion. For small focal lesions with an identifiable and sizeable feeding vessel, subselective catheterization was sometimes attempted in both groups. A CT scan was performed after treatment to document tumor coverage with Lipiodol and confirm that the whole tumor has been adequately treated. Additional Lipiodol-ethanol treatment or chemoembolization were given to the patients when there was CT evidence of residual or recurrent viable tumor depicted as contrast enhanced lesions at the arterial phase. All CT reports were prepared by independent radiologists.

Result

When comparing Lipiodol-ethanol to chemoembolization, the degree of Lipiodol retention in tumor at two months was significantly higher in the Lipiodol-ethanol group than in the chemoembolization group (89.5±10.7% versus 47.5±21.2%, P<0.0001) (table 1) Tumor response to treatment at 1 year or less-expressed in a ratio of number of patients showing partial response to static disease to lesion-size progression in the two groups were 18:12:0 (Lipiodol-ethanol group) and 12:13:5 (chemoembolization group). When the two groups were compared in terms of proportion of patients with lesion-size progression to that of non-progression, the ratio were 0:30 (Lipiodol-ethanol group) and 5:25 (chemoembolization group) (P=0.0261, Fisher exact test) (table 2), indicating a significantly higher chance of patients with lesion-size progression in the chemoembolization group. The 1 year and 2 year overall survival rate in the Lipiodol-ethanol group (93.3%, 80.0%) were significantly higher than those in the chemoembolization group (73.3%, 43.3%) (P=0.0053) (table 2). Extrahepatic disease progression of the Lipiodol-ethanol group at 1 year and 2 year (0%, 0%) were significantly lower than those in the chemoembolization group (35.5%, 39.2%) (P=0.0002) (table 2). There was no statistically significant difference in intrahepatic disease progression at 1 year and 2 year between the Lipiodol-ethanol group (30.2%, 41.2%) and the chemoembolization group (44.3%, 48.0%) (P=0.2613) (table 2). There was also no statistically significant difference in the progression free survival rate at 1 year and 2 year between the Lipiodol-ethanol group (69.8%, 58.8%) and the TACE group (46%, 42.5%) (P=0.0588) (table 2).

When considering all 60 patients as a whole group, it was found that patients with a higher degree of Lipiodol retention in their treated tumors (>60%) were associated with significantly better clinical outcome at 1 year than those with degree of Lipiodol retention □60%, in terms of a higher overall survival rate (88.9% versus 66.7%, P=0.0192), a lower intrahepatic disease progression rate (25.6% versus 59.4%, P=0.0169), a lower extrahepatic disease progression rate (0.31% versus 35.5%, P=0.0047), and a higher progression free survival rate (72.1% versus 36.3%, P=0.005) (table 3).

The overall survival period was prolonged from a mean of 1.28±0.13 years (□60% Lipiodol retention) to 2.61±0.16 years (>60% Lipiodol retention). Time to intrahepatic disease progression was prolonged from a mean of 0.70±0.10 years (D60% Lipiodol retention) to 1.41±0.10 years (>60% Lipiodol retention). Progression free survival period was prolonged from a mean of 0.63±0.1 years (□60% Lipiodol retention) to 1.38±0.10 years (>60% Lipiodol retention).

Discussion

The goals of chemoembolization are to deliver a highly concentrated dose of chemotherapy to tumor cells, to prolong the contact time between the chemotherapeutic agents and the tumor cells, and to minimize systemic toxicity from the chemotherapeutic agents (16). Lipiodol is a key ingredient in the treatment protocol of chemoembolization; it serves the functions of a drug-carrying, tumor-seeking, as well as embolizing agent. The deliverable of chemoembolization is prepared in the form of an emulsion by mixing Lipiodol with an equal volume of drug-containing aqueous solution. Embolization of the tumor-feeding arterial branches in the procedure of chemoembolization is achieved partly by the infusion of Lipiodol-containing emulsion and partly by the administration of embolic agents such as gelatin sponge pledgets. As embolization is a vital part of the chemoembolization treatment (17-20), the efficacy of embolization is a crucial factor to treatment efficacy.

The formulation of Lipiodol-ethanol with a lowered proportion by volume of Lipiodol (33%) is designed to facilitate thorough infiltration of tumor vasculature by Lipiodol-ethanol while maintaining the potency of the embolization power of absolute ethanol in the target vasculature since it is not diluted by aqueous solution. Theoretically, the preserved embolization potency of Lipiodol-ethanol allows it to embolize portal venules effectively when the Lipiodol-ethanol passes to portal venules through the peribiliary plexus (10, 11), as a result, effective embolization of portal venules surrounding the tumor blocks off portal blood supply to tumor periphery and prevents the infused agent in tumor vasculature from draining away through the portal venules (7). If the above hypothesis is valid, the embolization efficacy of Lipiodol-ethanol is likely to be superior to that of ethanol-free Lipiodol formulations such as that being used for chemoembolization. The authors believe transarterial ethanol ablation (TEA) has not been utilized to the extent that chemoembolization (TACE) is utilized in the West, because: 1) the promising preliminary results of TACE when it was introduced have created an overwhelming response and favorable attraction to the procedure among active medical researchers and practitioners in the past one and a half decades, so much so that TEAT has not gained much attention, 2) TEA requires a more tedious procedure of subselective catheterization, 3) the lack of chemoembolization agent in TEA treatment is less persuasive as an anti-cancer treatment when compared with TACE.

The possibility that ischemia and hypoxia may be a potent stimulator of angiogenesis and carcinogenesis in the liver (21, 22) has created doubts on treatment like transarterial embolization for HCC, however, the goal of transarterial ethanol ablation with Lipiodol-ethanol is not to achieve tumor ischemia, it is rather to completely occlude the vasculature of tumor lesions and thereby to achieve infarction of the lesions. Moreover, when the tumor vasculature is infiltrated with Lipiodol-ethanol, diffusion of ethanol from tumor vasculature into tumor tissue is likely to occur. It has been shown in several studies that percutaneous injection of ethanol into tumor interstitium and chemoembolization may have a synergistic effect that enables effective treatment of large liver tumors (23). Chemoembolization is known to enhance diffusion of ethanol within tumor tissue and achieve complete tumor necrosis (24, 25), resulting in significantly prolonged patient survival than that due to ethanol injection alone (24, 27). Given the observation on the enhanced treatment effects of combined chemoembolization and ethanol injection, the component of ethanol in the treatment of Lipiodol-ethanol is likely to provide the added advantage of ethanol ablation to the tumor in addition to the treatment effects of transarterial embolization.

The formulation of chemoembolization employed in the current study consisted of 10 mg of cisplatin per 20 ml of Lipiodol-aqueous emulsion, in which the dosage of chemotherapeutic drug is lower than that commoner used in some countries such as the United States, where the dosage of cisplatin used is up to 150 mg per 20 ml emulsion. However, the dosage of cisplatin in chemoembolization used in some studies that have shown the effectiveness of chemoembolization has also been as low as 10 mg per 20 ml emulsion (4, 28). There is no evidence on the requirement of a certain dose threshold of chemotherapeutic drug for effective chemoembolization treatment. The optimal dose of chemotherapeutic drug required for effective chemoembolization treatment is yet to be determined.

Effective embolization of arterial tumor feeders and tumor vasculature logically occlude vasculature within the tumor and prevent washout of Lipiodol from the tumor, degree of Lipiodol retention therefore is a reasonable indicator of embolization efficacy. Degree of Lipiodol retention as evaluated with non-enhanced CT has been used for quantitative assessment of the results of embolization in chemoembolization (29). A high degree of Lipiodol retention has been shown to be associated with a significantly prolonged median survival (29) and a greater extent of tumor necrosis as a result of chemoembolization (30). The degree of Lipiodol retention can be assessed mechanically with CT and therefore serves as an objective parameter for comparison between the two groups.

The results of the current study show that embolization efficacy of Lipiodol-ethanol in terms of degree of Lipiodol retention is superior to that of chemoembolization. The treatment efficacy of Lipiodol-ethanol in terms of tumor response and patient outcomes is superior to that of chemoembolization. The results also show that embolization efficacy as represented by the degree of Lipiodol retention is a useful early predictor of the treatment efficacy of Lipiodol-based transarterial treatments. The 1-year and 2-years survival rate of patients treated with Lipiodol-ethanol in our study (93.3% and 80.0%) were apparently more promising than those of patients treated with transarterial embolization using Gelfoam fragments in Llovet's study (75% and 50%) (3). Tumor characteristics in terms of lesion multiplicity and mean lesion size (52 mm, 52 mm) were identical in both studies. The 1-year and 2-years survival rates associated with chemoembolization in our study (73.3% and 43.3%) compared favorably with those in Lo's study (57% and 31.4%) (4), although they were slightly less favorable when compared with those in Llovet's study (3).

The occurrence of new intrahepatic tumors is relatively independent to the result of treatment to existing tumors, it is an indicator of the underlying liver condition.

Molecular studies have shown that recurrence after resection has two components. The main component, which occurs mainly within the first 2 years after resection (31), represents true metastasis that results from HCC dissemination before resection and is undetectable by imaging techniques (32). The other component includes metachronous tumors that arise de novo in a preneoplastic cirrhotic liver (33).

The parameter of intrahepatic disease progression used in the current study is an indicator of occurrence of new intrahepatic lesions. The fact that there was no difference in intrahepatic disease progression rate between the Lipiodol-ethanol group and chemoembolization groups indicated that there was no difference in the underlying liver condition in patients of the two groups. The occurrence of extrahepatic disease reflects a failure of control of intrahepatic lesions that subsequently invade extrahepatic structures, it is therefore an indicator of treatment efficacy to the intrahepatic tumor. The fact that there was a significantly higher rate of extrahepatic disease progression in the chemoembolization group indicated that treatment efficacy of chemoembolization is inferior to that of the Lipiodol-ethanol.

Further analysis of the study result by correlating the degree of Lipiodol retention to treatment efficacy from the perspective of the whole group of 60 patients provided a cross checking to the result of comparison between the two subgroups. The result of such further analysis confirmed the correlation between the degrees of Lipiodol retention to treatment efficacy; it was consistent with the result of comparison between the two subgroups, in that the degree of Lipiodol retention was higher in the Lipiodol-ethanol group while the treatment efficacy was also higher in the Lipiodol-ethanol group. A study of the correlation between the degrees of Lipiodol retention to treatment efficacy within each subgroup was not carried out, because the number of subject within the subgroup was not sufficient and the variation of degree of Lipiodol retention within the Lipiodol-ethanol group was too small.

Potential toxicities to normal liver may include ischemic damage to normal hepatocytes leading to atrophy of liver parenchyma. However, with selective catheterization to avoid excessive delivery of Lipiodol-ethanol to normal liver, complications of transarterial treatment such as acute hepatic decompensation and irreversible hepatic decompensation (8.6% and 0.6%) occurred much less frequently with Lipiodol-ethanol treatment (15) when compared to what was observed with chemoembolization (20%, 3%) in the literature (34). Other known complications of chemoembolization such as gastrointestinal bleeding from peptic ulcer or gastritis, hepatic encephalopathy, variceal bleeding, liver abscess, acalculous cholecystitis, and liver abscess did not occur with Lipiodol-ethanol treatment in the current study or in a previous study (31).

The number of patients recruited in this study is sufficient to show a significant difference in embolization efficacy and treatment efficacy between the two groups. The duration of follow up is adequate for observation of one year and two year survival rates, and is conclusive in showing a significant difference in clinical outcome in favor of the Lipiodol-ethanol group, such that a longer duration of follow up is not necessary.

The findings suggest that embolization efficacy and treatment efficacy of transarterial ethanol ablation with Lipiodol ethanol mixture is superior to that of chemoembolization for the treatment of patients with unresectable HCC. Embolization efficacy as represented by the degree of Lipiodol retention is a useful early predictor of treatment efficacy.

Further to the above, alternative embodiments of the present invention has also shown signs of success. In one alternative embodiment, the present invention for the treatment of neoplastic areas or solid tumors by occluding the blood supply to the tumor comprise transarterial administration of a fluid formulation comprising of absolute ethanol and a combination of fatty acids such as linolenic acid, linoleic acid, and oleic acid. This formulation comprising ethanol and the combination of fatty acids is herein identified as EFA. The EFA formulation comprises of about 20% to about 50% of absolute ethanol by volume and improves upon the ethanol's therapeutic attributes.

Research shows that this embodiment is a transarterial therapeutic agent for cancer treatment and serves three basic purposes including embolic agent to block off the arterial supply and venous pathways connected to the tumor, sclerosing agent to induce inflammation and fibrosis within the tumor vasculature, as well as a chemical ablative agent on direct contact to cause cell membrane destruction and protein denaturation and thereby leading to death of the tumor cells.

Beyond the above disclosed therapeutic attributes, the ethanol in this EFA formulation also acts as a solvent to the combination of fatty acids so that the viscosity of the final formulation is reduced to facilitate infiltration into minute tumor vessels and sinusoidal spaces within tumor substance. The combination of fatty acids serves to dilute the thrombogenic and cytotoxic effect of absolute ethanol on the endothelium of blood vessels leading to the tumor, so that the delivery of the formulation to the tumor is not hindered by thrombosis of these vessels.

Another function of the EFA formulation is to also treat solid tumors by transarterial administration of the formulation as a carrying agent of other chemotherapeutic agents so that these agents are retained in the solid tumors for a longer period of time. Because of the embolic and sclerosing action of the formulation, arteries and veins connected to the tumor are blocked by the formulation due to thrombosis and inflammatory response, incoming blood to the tumor and blood draining away from the tumor are shut down, so that the formulation itself and the chemotherapeutic agents carried with it are retained within the tumor. Clinical studies have shown that the formulation can be retained within tumors for a period of 15 to 51 months. Pharmacokinetic studies have shown that the drug retention ability of this formulation is better than that of oil-aqueous emulsion, and comparable to that of drug-eluting beads.

One embodiment of the therapeutic composition is the combination of approximately 10 cc EFA with approximately 20 mg of cisplatin powder with diameter ranging from approximately 5-50 micrometer, whereby up to approximately 50 cc of EFA and approximately 100 mg of cisplatin powder my be given safely within one treatment session. Although, cisplatin is a known chemotherapeutic agent, it has never been combined with the EFA formulation discussed in the present invention. Furthermore, typical particulate drug-carriers containing cisplatin utilizes particle sizes of around 300 micrometers to prevent the cisplatin from diluting or washing away. However the larger particle size also prevents the cisplatin from entering minute tumor vessels.

In the present invention, the smaller size of the cisplatin powder (5-50 micrometer) allows the resultant mixture to be effectively carried into minute tumor vessels and sinusoidal blood spaces within tumor tissue. Histological examination of tumor specimen obtained after treatment with EFA showed infiltration of EFA into the full thickness of tumor capsule, indicating that tumor islands entrapped within tumor capsule can be reached by EFA and the therapeutic agents carried with it. Tumor islands within tumor capsule normally cannot be reached by formulations other than EFA, especially the particulate drug-carriers, therefore these tumor islands normally cannot be effectively treated. Furthermore, it is found that due to the dual embolization effect of EFA in the arterial system as well as portal venous system, the EFA prevents the smaller cisplatin powder from diluting or washing away thus leading to a better overall retention rate in the tumor vasculature. In the present invention, the cisplatin powder and the EFA are usually retained for months (up to 51 months) within the tumor vasculature, though effectively drug retention for a lesser duration is still sufficient to achieve therapeutic purpose. Lastly, the smaller cisplatin powder in the present invention is released in a more sustained and slow fashion than the prior art. Smaller particle sizes of other therapeutic agents may also be effectively combined with the EFA disclosed in the present embodiment.

Another embodiment of the present invention is to treat solid tumors by transarterial administration of the EFA formulation as a carrying agent of paramagnetic nanoparticles or microparticles such as iron oxide, ferromagnetic nanoparticles or microparticles such as iron, or biologically-inert metallic nanoparticles or microparticles such as tantalum. These particles can be activated by external fields of electromagnetic wave or magnetic field to a state of hyperthermia so that they serve as a form of localized hyperthermia therapy. Because hyperthermic cytotoxicity is enhanced within a microvascular environment which has become acidic and hypoxic as a result of embolization, the therapeutic effect of local hyperthermia is theoretically ideal in the microvascular environment created in tumor embolization. Simultaneous localized hyperthermia and embolization is made possible with the administration of the current formulation.

The EFA disclosed in the present invention is well suited as a carrying agent of the above thermogenic particles as well as other chemotherapeutic agents. Within a microvascular environment consisting of high local concentration of retained chemotherapeutic agent, localized hyperthermia at moderate temperatures can interact with chemotherapeutic agents to result in synergistic cytotoxic effect on tumors. Experiments have shown that when the tumor vasculature is filled up with the formulation that consists of 1% of thermogenic particles by volume, the local temperature within the tumor can be elevated to 45 degree Celsius when the thermogenic particles are activated with an external electromagnetic wave at frequency 920 to 925 MHz and power 900 Watt for 25 minutes. Further temperature rise could be achieved by varying the amount of thermgenic paticles, and increasing the power and duration of external electromagnetic wave.

In one embodiment of the present invention, the resultant mixture comprise of approximately 10 cc EFA mixed with approximately 0.1 cc (0.52 gm) of iron oxide nanoparticles of diameter 20-30 nanometers and delivered to capillaries and arterioles of tumor vasculature by EFA. In this EFA-iron oxide therapeutic composition, a total of approximately 50 cc EFA and approximately 0.5 cc (2.6 gm) of iron oxide nanoparticles can be given safely within one treatment session. The iron oxide nanoparticles are retained in tumor vasculature by EFA for a prolonged period due to the dual embolization effect of EFA in the arterial system as well as portal venous system. When a tumor of approximately 50cc volume, with approximately 20% of its volume consisting of vasculature, has its vasculature completely filled with the EFA-iron oxide formulation, a temperature rise of to approximately 45 degree Celsius can be achieved.

In yet another embodiment, tantalum nanoparticles or microparticles may also be added to form a resultant mixture comprising (1) ethanol; (2) mixture of linolenic acid, linoleic acid, and oleic acid; and (3) tantalum particles. The addition of tantalum particles is distinguishable due to its ability to act as both a contrast agent and a hyperthermia therapy agent.

TABLE 1
Patient and Tumor Characteristics and Comparison
of Embolization Efficacy between LEM and TACE

| | LEM | TACE | P-value |
|---|---|---|---|
| Patient number | 30 | 30 | |
| Patient age | 64.4 ± 11.2 | 62.7 ± 10.8 | 0.544 |
| Sex ratio (M:F) | 24:6 | 23:7 | 0.754 |
| Greatest tumor dimension (mm) | 52.1 ± 22.8 | 57.6 ± 25.5 | 0.382 |
| Child-Pugh Classification A:B | 28:2 | 28:2 | 1.00 |
| ECOG grade ≦1:2 | 30:0 | 29:1 | 0.99 |
| Percentage lipiodol retention | 89.5 ± 10.7 | 47.5 ± 21.2 | <0.0001 |
| at 2 months after first | | | |
| treatment | | | |

Footnote:
ECOG represents Eastern Cooperative Oncology Group Performance Status

TABLE 2
Comparison of Treatment Efficacy between LEM and TACE

| | LEM | TACE | P-value |
|---|---|---|---|
| Tumor response by RECIST (30 patients) | 30:0 | 25:5 | 0.0261 |
| Non-progressive lesion:Progressive lesion | | | |
| Overall survival (%) | | | 0.0053 |
| 1 year | 93.3 | 73.3 | |
| 2 year | 80.0 | 43.3 | |
| Intrahepatic disease progression rate (%) | | | 0.2613 |
| 1 year | 30.2 | 44.3 | |
| 2 year | 41.2 | 48.0 | |
| Progression free survival rate (%) | | | 0.0002 |
| 1 year | 0 | 35.5 | |
| 2 year | 0 | 39.2 | |
| Progression free survival rate (%) | | | 0.0588 |
| 1 year | 69.8 | 46.0 | |
| 2 year | 58.8 | 42.5 | |

TABLE 3
Correlation Between Embolization Efficacy
and Treatment Efficacy of the Whole Group

| | Percentage | | | | 95% C.I. |
|---|---|---|---|---|---|
| | Retention | Lipiodol | P- | Hazard | of Hazard |
| End-point | <60%> | 60% | value | Ratio | Ratio |
| One year | 66.7% | 88.9% | 0.0192 | 0.370 | 0.161-0.850 |
| survival rate | | | | | |
| One year | 59.4% | 25.6% | 0.0169 | 0.384 | 0.167-0.886 |
| Intrahepatic | | | | | |
| disease | | | | | |
| progression | | | | | |
| rate | | | | | |
| One year | 35.5% | 0.31% | 0.0047 | 0.051 | 0.067-0.403 |
| extrahepatic | | | | | |
| disease | | | | | |
| progression | | | | | |
| rate | | | | | |
| One year | 36.3% | 72.1% | 0.0050 | 0.347 | 0.165-0.725 |
| progression | | | | | |
| free survival | | | | | |
| rate | | | | | |

Although only preferred embodiments are specifically disclosed and claimed herein, it will be appreciated that further modifications of the invention may be made without departing from the spirit and intended scope of the invention.

### REFERENCES

1. Kojiro M, Nakashima T. Pathology of hepatocellular carcinoma. In: Okuda K, Ishak K G, eds. Neoplasms of the liver. Tokyo, Japan: Springer-Verlag, 1987; 81-140.

2. Matsuyi O, Kadoya M, Yoshikawa J, et al. Small hepatocellular carcinoma: treatment with subsegmental transcatheter arterial embolization. Radiology 1993; 188: 79-83.

3. Bronowicki J P, Vetter D, Doffoel M: Chemoembolization of hepatocellular carcinoma. In; Okuda K, Tabor E, eds. Liver cancer. New York. Churchill Livingstone, 1997, 463469.

4. Kan Z, Ivancev K, Lunderquist A. Peribiliary plexa-important pathways for shunting of iodized oil and silicon rubber solution from the hepatic artery to the portal vein: an experimental study in rats. Invest Radio 1994; 29: 671-676.

5. Kan Z, Wallace S. Transcatheter liver lobar ablation: an experimental trial in an animal model. Eur Radiol. 1997; 7: 1071-1075.

6. Park J H, Han J K, Chung J W, Choi B I, Han M C, Kim Y I. Superselective transcatheter arterial embolization with ethanol and iodized oil for hepatocellular carcinomd. J Vascu Intery Radiol. 1993; 4; 333-339.

7. Matsui 0, Kadoya M, Yoshikawa J, et al. Small hepatocellular carcinoma: treatment with subsegmental transcatheter arterial embolization. Radiology 1993; 188: 79-83.

8. Ito K, Kusunoki H, Okamoto E, et al. Intra-arterial alcoholization of advanced hepatocellular carcinoma. Can Chemo Pharmacol 1994; 33 (S): S 42-5 47.

9. Kojiro M, Nakashima T. Pathology of hepatocellular carcinoma. In: Okuda K: Ishak K G, eds. Neoplasms of the liver. Tokyo, Japan: Springer-Verlag, 1987; 81-104.

10. Matsui O, Kadoya M, Kameyama T, et al. Benign and malignant nodules in cirrhotic livers: distinction based on blood supply. Radiology 1991; 178: 493̂-97.

11. Ueno K, Miyazono N, Inoue H, Nishida H, Kanetsuki I, Nakajo M. Transcatheter arterial chemoembolization therapy using iodized oil for patients with unresectable hepatocellular carcinoma: evaluation of three kinds of regimens and analysis of prognostic factors. Cancer 2000; 88: 1574-1581.

12. Ngan H, Lai C L, Fan S T, Lai E C S, Yuen W K, Tso W K. Treatment of inoperable hepatocellular carcinoma by transcatheter arterial chemoembolization using an emulsion of cisplatin in iodized oil and gelfoam. Clin Radiol 1993; 47: 315-320.

13. Takayasu K, Moriyama N, Muramatsu Y, et al. Gallbladder infarction after hepatic artery embolization. Am J Roentgenol. 1985; 144:135-138.

14. Group d'Etude et de treatment du carcinoma hepatocellulaire. A comparison of contrast/carrier agent chemoembolization and conservative treatment for unresectable hepatocellular carcinoma. N Engl J Med. 1995; 332: 1256-1261.

15. El-Serag H B, Mason A C. Rising incidence of hepatocellular carcinoma in the United States. N Engl J Med 1999; 340: 745-750.

16. Taylor-Robinson S D, Thomas H C, Arora S, Hargreaves S. Increased mortality from liver cancer in England and Wales is not related to hepatitis C (letter). BMJ 1999; 319: 640,

17. Llovet J M, Real M I, Montana X, et al. Arterial embolisation or chemoembolisation vs symptomatic treatment in patients with unresectable HCC: a randomized controlled trial. Lancet 2002; 359: 1734 -1739.

18. Lo C M, Ngan H, Tso W K, et al. Randomized controlled trial of Transarterial Lipiodol Chemoembolization for unresectable HCC. Hepatology 2002; 35: 1164-1171.

19. Maluccio M A, Covey A M, Porat L B, et al. Transcatheter arterial embolization with only particles for the treatment of unresectable hepatocellular carcinoma. J Vase intery Radiol. June 2008: 19(6): 862-9. Epub Apr. 10, 2008.

20. Sato K T, Lewandowski R J, Mulcahy M F, et al. Unresectable chemorefractory liver metastases: radioembolization with 90Y microspheres—safety, efficacy and survival. Radiology 2008; 247(2): 507-15.

21. Matsui 0, Kadoya M, Yoshikawa J, et al. Small hepatocellular carcinoma: treatment with subsegmental transcatheter arterial embolization. Radiology 1993; 188: 79-83

22. Park J H, Han J K, Chung J W, Choi B I, Han M C, Kim Y I. Superselective transcatheter arterial embolization with ethanol and iodized oil for hepatocellular carcinoma. J Vascu Intery Radiol. 1993; 4; 333-339.

23. Ito K, Kusunoki H, Okamoto E, et al. Intra-arterial alcoholization of advanced hepatocellular carcinoma. Can Chemo Pharmacol 1994; 33(S): S 42-S 47.

24. Kan Z, Ivancev K, Lunderquist A. Peribiliary plexa-important pathways for shunting of iodized oil and silicon rubber solution from the hepatic artery to the portal vein: an experimental study in rats. Invest Radiol 1994; 29: 671-676.

25. Kan Z, Wallace S. Transcatheter liver lobar ablation: an experimental trial in an animal model. Eur Radiol. 1997; 7: 1071-1075.

26. Cheng Y, Kan Z, Chen C, et al. Efficacy and safety of preoperative lobar or segmental ablation via transarterial administration of ethiodol and ethanol mixture for treatment of hepatocellular carcinoma: clinical study. World J Surg. 2000; 24(7): 844-50; discussion 850.

27. Cheung Y C, Ko S F, Ng S H, Chan S C, Cheng Y F. Survival outcome of lobar or segmental transcatheter arterial embolization with ethanol-lipiodol mixture in treating hepatocellular carcinoma. World J Gastroenterol. 2005; 11(18): 2792 -2795.

28. Miller A B, Hoogstraten B, Staquet M, Winkler A. Reporting results of cancer treatment. Cancer 1987; 47(1): 207-214.

29. Yu S C H, Hui E P, Wong J, et al. Transarterial ethanol ablation of hepatocellular carcinoma with lipiodol-ethanol mixture: Phase II Study. J Vase intery Radiol. 2008; 19. 95-103.

30. Ramsey D E, Kemagis L Y, Soulen M C, Geschwind J H. Chemoembolization of Hepatocellular Carcinoma. J Vas Intery Radiol 2002; 13: S211-S221.

31. Koiino T. Targeting cancer chemotherapeutic agents by use of lipiodol contrasat medium. Cancer 1990; 66: 1897-1903.

32. Egawa H, Maki A, Mori K. Effects of intraartcrial chemotherapy with a new lipophilic anticancer agent, estradiolchlorambucil (KM2210), dissolvded in lipiodol on experimental liver tumor in rats. J Surg Oncol 1990; 44: 109-114.

33. Nakamura H, Hashimoto T, Oi H, Sawada S. Transcatheter oily chemoembolization of hepatocellular carcinoma. Radiology 1989; 170: 783-786.

34. Sasaki Y, Imaoka S, Kasugai H, et al. A new approach to chemoembolization therapy for hepatoma using ethiodized oil, cisplatin, and gelatin sponge. Cancer 1987; 60: 1194-1203.

35. Mathupala S P, Rempel A, Pedersen P L. Glucose catabolism in cancer cells: identification and characterization of a marked activation response of the type II hexokinase gene to hypoxic conditions. J Biol Chem 2001; 276: 43407-43412.

36. Tajima T, Honda H, Taguchi K, et al. Sequential hemodynamic change in hepatocellular carcinoma and dysplastic nodules: CT angiography and pathologic correlation. AJR Am J Roentgenol 2002; 178: 885-897.

37. Tanaka K, Okazaki H, Nakamura S, et al. Hepatocellular carcinoma: treatment with a combination therapy of transcathcter arterial embolization and percutaneous ethanol injection. Radiology 1991; 179: 713-717.

38. Lencioni R, Vignali C, Caramella D, Cioni R, Mazzeo S, Bartolozzi C. Transcatheter arterial embolization followed by percutaneous ethanol injection in the treatment of hepatocellular carcinoma. Cardiovas Intery Radiol 1994; 1: 70-75.

39. Tanaka K, Nakamura S, Numata K, et al. Hepatocellular carcinoma; treatment with percutaneous ethanol injection and transcatheter arterial embolization. Radiology 1992; 185:457-460.

40. Hasuike Y, Okamura J, Furukawa J, et al. Efficacy of combination treatment (TAE with adriamycin and ethanol) for hepatocellular carcinoma. Cancer Chemother Pharmacol 1992; 31(suppl):S30-S34.

41. Bartolozzi C, Lencioni R, Caramella D, et al. Treatment of large HCC: transcatheter arterial chemoembolization combined with percutaneous ethanol injection versus repeated transcatheter arterial chemoembolization. Radiology 1995; 197: 812-818.

42. Ngan H, Lai C L, Fan S T, Lai E C, Yuen W K, Tso W K. Treatment of inoperable hepatocellular carcinoma by transcatheter arterial chemoembolization using an emulsion of cisplatin in iodized oil and gelfoam. Clin Radiol. 1993; 47: 315-320.

43. Vogl T J, Trapp M, Schroeder H, et al. Transarterial chemoembolization for hepatocellular carcinoma: volumetric and morphologic CT criteria for assessment of prognosis and therapeutic success-results from a liver transplantation center. Radiology. 2000; 214(2): 349-357.

44. Choi B J, Kim H C, Han J K, et al. Therapeutic effect of transcatheter oily chemoembolization therapy for encapsulated nodular hepatocellular carcinoma: CT and pathological findings Radiology 1992; 182: 709-713.

45. Imamura H, Matsuyama Y, Tanaka E, eta al. Risk factors contributing to early and late phase intrahepatic recurrence of hepatocellular carcinoma after hepatectomy. J Hepatol. 2003; 38(2): 200-207.

46. Llovet J M, Schwartz M, Mazzaferro V. Resection and liver transplantation for hepatocellular carcinoma. Semin Liver Dis. 2005; 25(2): 181-200.

47. Llovet Jm, Di Bisceglie A M, Bruix J, et al. Design and Endpoints of Clinical Trials in Hepatocellular Carcinoma. J Natl Cancer Inst 2008; 100: 698-711.

48. Chan A O, Yuen M F, Hui C K, Tso W K, Lai Cl. A prospective study regarding the complications of transcatheter intraarterial lipiodol chemoembolization in patients with hepatocellular carcinoma. Cancer 2002: 94: 1747-1752.

What is claimed:

1. A therapeutic composition for treatment of hepatic solid tumors comprising a fatty acid mixture and ethanol wherein the ratio of said fatty acid mixture to ethanol is about 1 to 1 to about 5 to 1 and maybe retained in said tumors from about 15 month to about 51 month.

2. The therapeutic composition for treating hepatic solid tumors of claim 1 wherein the said fatty acid mixture is a combination of fatty acids comprising of linolenic acid, linoleic acid, and oleic acid.

3. The therapeutic composition for treating hepatic solid tumors of claim 1 further comprising a therapeutic agent which may comprise of cisplatin, paclitaxol, doxorubicin, and/or ethanol.

4. The therapeutic composition for treating hepatic solid tumors of claim 1 further comprising a hyperthermia therapy agent.

5. The therapeutic composition for treating hepatic solid tumors of claim 4 wherein said hyperthermia therapy agent comprise of iron oxide nanoparticles with a diameter of approximately 20 to 30 nanometers.

6. The therapeutic composition for treating hepatic solid tumors of claim 4 wherein said hyperthermia therapy agent comprise of tantalum nanoparticles or microparticles.

7. The therapeutic composition for treating hepatic solid tumors of claim 1 wherein the said composition may be administered to a solid tumor by intra-arterial injection that carries blood to the tumor and is retained in a solid tumor, occludes arterial vasculature of said solid tumor and occludes portal venous vessels that supply liver tumors.

8. The composition for treating hepatic solid tumors of claim 1 where in the said therapeutic composition serves as a embolic agent, sclerosing agent and a chemical ablative agent.

9. A method of treating hepatic solid tumors by administering a therapeutic composition to the solid tumors comprising:
combining a therapeutic agent with a mixture of fatty acid mixture and ethanol to form said therapeutic composition,
administering said therapeutic composition by intra-arterial injection into an artery that carries blood to said tumors,
wherein the ratio of said fatty acid mixture to ethanol is about 1 to 1 to about 5 to 1 and the retention time of said therapeutic composition in said tumors is from about 15 month to about 51 month.

10. The method according to claim 9 wherein said therapeutic agent may comprise of cisplatin, paclitaxol, doxorubicin, and/or ethanol.

11. The method according to claim 10 wherein said therapeutic agent comprise cisplatin powder with a diameter of 5-50 micrometer.

12. The method according to claim 11 wherein approximately 20 mg of said cisplatin powder is mixed with approximately 10 cc of said combined fatty acid mixture and ethanol.

13. The method according to claim 9 wherein a hyperthermia therapy agent is mixed into the said therapeutic composition prior to administration.

14. The method according to claim 13, wherein said hyperthermia therapy agent comprise of iron oxide nanoparticles with a diameter of approximately 20 to 30 nanometers or tantalum nanoparticles or microparticles.

15. The method according to claim 9, wherein said resultant mixture accumulates and is retained in a solid tumor, occludes arterial vasculature of said solid tumor and occludes portal venous vessels that supply liver tumors.

16. The method according to claim 9 wherein the said fatty acid mixture is a combination of fatty acids comprising of linolenic acid, linoleic acid, and oleic acid.

17. A method of delivering a therapeutic composition capable of infiltrating minute tumor vessels to hepatic solid tumors comprising:
administering a mixture of a fatty acid mixture, ethanol, a therapeutic agent and a thermotherapy agent by intra arterial injection into an artery that carries blood to the hepatic tumors wherein the ratio of said fatty acid mixture to ethanol is about 1 to 1 to about 5 to 1 and the retention time of the mixture and thermotherapy agent is about 15 to 51 month.

18. The method according to claim 17 wherein the said hyperthermia therapy agent comprise of iron oxide nanoparticles of approximately 20 to 30 nanometers in diameter and wherein the ratio is approximately 0.1 cc of said iron oxide nanoparticles to every approximately 10 cc of combined fatty acid mixture and ethanol.

19. The method according to claim 17 wherein the said therapeutic agent comprise of cisplatin powder of approximately 5-50 micrometer in diameter and approximately 20 mg of cisplatin powder is added for every approximately 10 cc of combined fatty acid mixture and ethanol.

20. The method according to claim 17 wherein the said fatty acid mixture is a combination of fatty acids comprising of linolenic acid, linoleic acid, and oleic acid and the said therapeutic composition serves as a embolic agent, sclerosing agent and a chemical ablative agent.
